(19) United States
(12) Patent Application Publication
(10) Pub. No.: US 2025/0235418 A1
(43) Pub. Date: Jul. 24, 2025

(54) Butyrate Analogues and Methods of Use for HIF Stabilization and Treatment of Bowel Disease

(71) Applicant: THE REGENTS OF THE UNIVERSITY OF COLORADO, A BODY CORPORATE, Denver, CO (US)

(72) Inventors: Sean P. COLGAN, Foxfield, CO (US); Ruth X. WANG, Denver, CO (US); Alfredo ORNELAS-SANCHEZ, Denver, CO (US)

(21) Appl. No.: 18/853,910

(22) PCT Filed: Apr. 4, 2023
(86) PCT No.: PCT/US2023/017501

§ 371 Date: Oct. 3, 2024

Related U.S. Application Data
(60) Provisional application No. 63/327,335, filed on Apr. 4, 2022.

(51) Int. Cl. A61K 31/19; A61K 31/192; A61K 31/197

(52) U.S. Cl. CPC A61K 31/19; A61K 31/192; A61K 31/197

(57) ABSTRACT

Compositions comprising butyric acid and derivatives thereof for a method of treating intestinal disease. These compositions act to stabilize HIF through the inhibition of PHDs.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates in general to inhibition of hypoxia-inducible factor (HIF) prolyl hydroxylases (PHDs) to stabilize HIF. The disclosure teaches butyrate, and derivatives thereof, to bind and function as a unique, noncompetitive inhibitor of PHDs relative to other Short-chain fatty acids (SCFAs). The disclosure teaches the use of HIF PHDs inhibitors for treatment and prevention of intestinal disease.

### BACKGROUND

The composition of the intestinal microbiota varies substantially between individuals and is thought to be a key determinant of host susceptibility to an increasing variety of diseases. In inflammatory bowel disease (IBD), which includes Crohn's disease and ulcerative colitis, the intestinal microbiota plays a key role in driving inflammatory responses during disease development and progression.

The exact cause of inflammatory bowel disease is unknown. Intestinal barrier dysfunction is directly linked to inflammatory bowel diseases. Gut barrier is regulated by a variety of microbial metabolites, including dietary fiber derived short chain fatty acids (e.g. butyrate). Intestinal barrier dysfunction is directly linked to inflammatory bowel diseases. Gut barrier is regulated by a variety of microbial metabolites, including dietary fiber derived short chain fatty acids (e.g. butyrate). In disease, butyrate producing bacteria are lost and butyrate transporters are downregulated. Butyrate (BA) regulates barrier in part through stabilization of hypoxia inducible factor (HIF). HIF-deficient IECs exhibit major defects in mucosal barrier integrity. In normoxia, HIF is targeted for degradation catalyzed by prolyl hydroxylase enzymes (PHD1-3), mainly PHD2.

There is significant interest in developing “well tolerated” small molecule inhibitors of PHDs to stabilize HIF in the treatment numerous disorders.

The embodiments disclosed herein are directed toward overcoming one or more of the problems discussed above.

### BRIEF SUMMARY

Various embodiments provide compositions and methods for inhibiting PHDs to stabilize HIF for the treatment of intestinal disease.

Analogues structurally related to butyrate more specifically stabilize HIF with a longer biological half-life. The strong association between the low pO₂ environment and the generation of large amounts of SCFAs in the colon, establishes the relationship between SCFAs and HIF stabilization. We teach butyrate, and derivatives thereof, is a direct, noncompetitive PHD inhibitor. We teach use of butyrate, and derivatives, thereof as an inhibitor of PHD for therapeutic treatment of intestinal disease.

In one embodiment, the composition for use in stabilizing HIF comprising 4-Mercaptobutyrate. In one embodiment, the composition is selected from the group consisting of Butyric acid, crotonic acid, 3-Chloro butyric acid, 2-Bromo butyric acid, 4-Mercapto butyric acid, GABA, 3-Phenyl butyric acid and combinations thereof.

In one embodiment, the composition is used to treat intestinal disease in a patient in need.

In one embodiment, a method of stabilizing HIF comprising administering to a subject in need a compound selected from the group consisting of Butyric acid, crotonic acid, 3-Chloro butyric acid, 2-Bromo butyric acid, 4-Mercapto butyric acid, GABA, 3-Phenyl butyric acid and combinations thereof.

In one embodiment, a method of treating intestinal disease comprising administering an effective amount of a compound selected from the group consisting of Butyric acid, crotonic acid, 3-Chloro butyric acid, 2-Bromo butyric acid, 4-Mercapto butyric acid, GABA, 3-Phenyl butyric acid and combinations thereof. In one embodiment, the subject has one or more symptoms of: Crohn's disease, ulcerative colitis, irritable bowel syndrome, inflammatory bowel disease, or gastrointestinal cancer.

In one embodiment, a method of treating or preventing abdominal pain or gastrointestinal pain in a subject, comprising administering to the subject a composition comprising 4-Mercaptobutyrate.

In one embodiment, a method to inhibit PHDs through administering a compound selected from the group consisting of Butyric acid, crotonic acid, 3-Chloro butyric acid, 2-Bromo butyric acid, 4-Mercapto butyric acid, GABA, 3-Phenyl butyric acid and combinations thereof.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 shows how MCPA inhibits B-oxidation of butyrate to diminish oxygen consumption. (A) Chemical structure of MCPA compared to butyrate. (B) Relative butyrate concentration remaining in media between 0 to 6 h of either 600 μM butyrate or 600 μM butyrate with 1 mM MCPA treatment in T84 cells (C) High performance liquid chromatography (HPLC) tracings of butyrate at 0 h and 6 h for 600 μM butyrate or 600 μM butyrate with 1 mM MCPA treatment in T84 cells. (D) Oxygen saturation of T84 cells treated with 5 mM butyrate with or without 1 mM MCPA over 30 min (E) Rates of oxygen consumption calculated from nonlinear regression of oxygen saturation data in T84 cells treated with 5 mM butyrate with or without 1 mM MCPA (F) Illustration of the mechanism of MCPA to inhibit β-oxidation of butyrate.

FIG. 2 shows butyrate stabilization of HIF in the presence of β-oxidation inhibition. (A) HIF-1a target mRNA expression in T84 cells treated with 10 mM butyrate with or without 1 mM MCPA, 1 mM DMOG, or 30 μM IOX2 for 4 h (B) HIF-1α protein expression in T84 cells treated with 10 mM butyrate with or without 1 mM MCPA for 4 h. (C) Quantified densitometry of HIF-1α protein expression in T84 cells treated with 10 mM butyrate with or without 1 mM MCPA for 4 h

FIG. 3 shows how PHD inhibitors and butyrate increase 2-OG. (A) PHD enzymatic reaction of 02 and 2-OG converted to CO2 and succinate in order to hydroxylate HIF-a proline residues. (B) 2-OG concentrations in T84 cells treated with 10 mM butyrate with or without 1 mM MCPA or 30 μM IOX2 for 3 h

FIG. 4 shows how recombinant PHD2181-402 expression and Michaelis-Menten constant (Km) determination of 2-OG for PHD2181-402. (A) PHD2181-402 compared to PHD1 (407 amino acid residues), PHD2 (426), and PHD3 (239), showing overall homology between the three human PHDs and PHD2181-402 spanning the highly conserved active site. (B) Size exclusion superdex 75 column elution of PHD2181-402. (C) 12% SDS gel image of the Superdex 75 column eluent confirming the expression and purity of PHD2181-402. (D) 100 nM of PHD2181-402 enzyme was incubated with various concentrations of substrate 2-OG from 625 nM to 40 μM, 10 μM HIF-1a547-581 peptide, 10 μM Fe (II), and 100 μM ascorbic acid and concentration of product succinate was measured after a 10-minute reaction to determine the rate

FIG. 5 shows how butyrate specifically and noncompetitively inhibits PHD2181-402. (A) Representative percentage of PHD2181-402 activity normalized to control based on succinate production following a 10-minute reaction of 1 μM PHD2181-402, 10 μM 2-OG, 10 μM Fe (II), 10 μM HIF-1a547-581 peptide (CODD), and 100 μM ascorbic acid in the presence of 195.3 μM to 100 mM SCFAs (acetate, propionate, butyrate, valerate, and hexanoate) and 78 μM to 40 mM DMOG (n=4, error bars: SEM, [Inhibitor] vs. normalized response-variable slope least squares fit). (B) True IC50 values for SCFAs and DMOG calculated from measured IC50 values accounting for percentage of substrate conversions between replicate experiments (n=5-12, error: SEM).

FIG. 6 shows NMR characterization of SCFAs binding to PHD2181-402. (A-D) WaterLOGSY ID NMR to determine SCFAs binding to PHD2181-402. 10 mM of each tested SCFA was mixed with 25 μM PHD2181-402. ((A) Butyric acid; peak inversions relative to the water signal were seen for protons directly bound to carbons C2 and C4, indicating binding. (B-D) Acetic acid, propionic acid and valeric acid, respectively; no peak is inverted, indicating no binding under these experimental conditions. (E-G) AFP-NOESY 1D NMR to determine butyrate atoms in the binding pocket of PHD2181-402. (E) The butyrate structure with labeled positions and corresponding arrow color code. (F) 10 mM butyrate was mixed with 25 μM PHD2181-402. The strength of the adiabatic pulse was gradually increased to shift relaxation contributions from longitudinal cross relaxation (NOESY) to transverse cross relaxation (ROESY). Protons attached to C4 were selectively inverted and acted as source of magnetization transfer. Peaks of protons attached to C2, unlike protons attached to C3, showed a profile typical for strong spin diffusion. This indicates embedding of the C2 in the binding pocket of the protein. (G) Protons attached to C2 were selectively inverted. Peaks of protons attached to C4, unlike protons attached to C3, showed a profile typical for strong spin diffusion, indicating embedding of the C4 protons in the binding pocket. The weak spin diffusion dependence of the protons attached to C3 in both (F) and (G) indicates that the C3 protons are more solvent exposed.

FIG. 7 shows how butyrate noncompetitively inhibits PHD2181-402. (A) True IC50 values for butyrate and DMOG in the presence of increasing 2-OG concentrations (n=4-6, error bars: SEM, ns not significant, *p<0.05, **p<0.01, ****p<0.0001 by 1-way ANOVA, Fisher's multiple comparison). (B) WaterLOGSY ID NMR of 10 mM butyrate incubated with 25 μM PHD2181-402 with and without 150 μM 2-OG. No peak inversion relative to the water signal was observed for any of the butyrate resonances indicating absence of binding in the presence of 2-OG. (C) Representative MST plot of 25 nM PHD2181-402 incubated with a range of butyrate concentrations from 9.2 μM to 75 mM with or without 500 nM 2-OG and assayed for protein and ligand interaction to determine binding affinity of butyrate to PHD2181-402. Measurements were repeated 3 times.

FIG. 8 shows how microbiota-derived butyrate inhibits PHDs in vivo. (A) HIF-1α target mRNA expression of Bnip31 in murine whole colon tissue and intestinal epithelial scrapings of control mice, mice treated with antibiotics (glycerol control), and mice given back tributyrin (200 μL, 3 d) with or without 60 mM MCPA for 4 h (B) HIF-1α protein expression in murine intestinal epithelial scrapings with corresponding quantified densitometry in mice treatment groups (C) HIF-1α protein expression in murine whole colon tissue with corresponding quantified densitometry in mice treatment groups (D) HIF-1α target mRNA expression in murine enteroids differentiated of epithelial lineage treated with 10 mM butyrate with or without 1 mM MCPA for 4 h (E) HIF-1α protein expression in murine enteroids treated with 10 mM butyrate with or without 1 mM MCPA for 4 h with corresponding quantified densitometry (F) Butyrate levels in murine whole colon tissue of mice treatment groups (G) 2-OG levels in murine whole colon tissue of mice treatment groups.

FIG. 9 shows how rectal gavage of butyrate inhibits PHDs in vivo. (A) HIF-la target mRNA expression in murine colon tissue of control mice, mice treated with antibiotics for microbiota and microbial-derived butyrate depletion, and mice rectally given back 100 mM butyrate, 100 mM butyrate with 10 mM MCPA, and 1.5 mM IOX2 for 1 h (B) 2-OG levels in murine colon tissue following antibiotics and rectal gavage treatments (C) HIF-1α protein expression in murine colon tissue. (D) Quantified densitometry of HIF-1α protein expression in murine colon tissue treated with rectal gavage of 100 mM butyrate with or without 10 mM MCPA or 1.5 mM IOX2 for 1 h.

FIG. 10 shows how microbiota-derived butyrate directly and noncompetitively inhibits PHDs to influence intestinal homeostasis. Butyrate regulates gene expression through HIF stabilization and impacts metabolite flux through elevating 2-OG levels.

FIGS. 11-20 various depictions of embodiments.

FIG. 21 shows MBA stabilization of HIF. (A) Structures of butyrate derivatives investigated in initial screen for HIF stabilization. Immunoblot of HIF1a protein levels in Caco-2 cells (B) and T84 cells (C) exposed to veh (HBSS+), 10 mM IOX4, or 5 mM butyrate derivatives for 6h. (D) Dose response study of HIF1a accumulation in T84 cells treated with decreasing concentrations of MBA and BA for 6 h.

FIG. 22 shows time course studies of MBA-HIF stabilization. (A) Immunoblots of HIF1a protein over time after exposure of T84 cells to 5 mM BA and MBA. (B) Mesoscale assay of HIF1a protein over time in caco-2 cells treated with 5 mM MBA compared to veh. (C) HIF1a and HIF target (BNIP3) protein in caco-2 cells exposed to veh (HBSS+), 10 mM IOX4 or 5 mM MBA. Data are presented as mean+S.E.M, *p<0.05, **p<0.01, ***p<0.001.

FIG. 23 shows how MBA transactivates HIF target genes. (A) Exposure of T84 cells to Veh, BA or MBA (5 mM, 16 h) induced HIF target gene expression of CAIX and BNIP3. (B) Influence of BA and MBA (5 mM, 16 h) on the expression of HIF targets in T84 cells lacking HIF1b (HIF1b KD) relative to non-targeting control lentivirus (sh Ctl). (C) Non-targeting control and T84 HIF1b depleted cells were analyzed for HIF1b expression by western blotting, shown with a representative blot. Quantification of HIF1b decrease was performed using ACTB-normalized densitometry. qPCR data was analyzed by One-way ANOVA with Bonferroni for post hoc correction and presented as mean±S.E.M, **p<0.01, ***p<0.001, ****p<0.0001 of three independent experiments.

FIG. 24 shows epithelial barrier formation response to MBA and BA. Barrier development in Caco-2 cells treated with Veh, BA or SHBA (5 mM); n=6. Data analyzed by Two-way ANOVA with Dunnett for post hoc correction and presented as mean±S.E.M, *p<0.05 **p<0.01, ***p<0.001, #BA, *MBA compared to veh in two independent experiments.

FIG. 25 shows how MBA induces HIF regulated targets in vivo. Erythropoietin (EPO) is regulated by HIF stabilization. (A) EPO protein in blood serum was assessed by mesoscale assay (n=6-8). qPCR mRNA expression of HIF target mGLUT1 in kidney (B) and colon tissue (C) (n=3-8). Data analyzed by One-way ANOVA with Tukey for post hoc correction and presented as mean±S.E.M of two independent experiments, *p<0.05, **p<0.01, ***p<0.001, ****p<0.0001 Erythropoietin (EPO) is regulated by HIF stabilization. (A) EPO protein in blood serum was assessed by mesoscale assay (n=6-8). qPCR mRNA expression of HIF target mGLUT1 in kidney (B) and colon tissue (C) (n=3-8). Data analyzed by One-way ANOVA with Tukey for post hoc correction and presented as mean±S.E.M of two independent experiments, *p<0.05, **p<0.01, ***p<0.001, ****p<0.0001

FIG. 26 shows a butyrate derivatives screen for HIF stabilization. HIF1a Mesoscale assay of BA derivatives investigated in caco-2 (A) and T84 (B) intestinal epithelial cells (5 mM, 6 h), identifying MBA as a promising candidate in HIF1a stabilization (Veh=HBSS+).

FIG. 27 shows epithelial barrier formation in the presence of butyrate derivatives. TEER of C2bbe cells exposed to 5 mM butyrate derivatives.

### DETAILED DESCRIPTION OF THE INVENTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Intestinal Disease includes but is not limited to: Crohn's disease, ulcerative colitis, irritable bowel syndrome, inflammatory bowel disease, or gastrointestinal cancer.

Butyrate and Butyrate derivatives include but are not limited to:

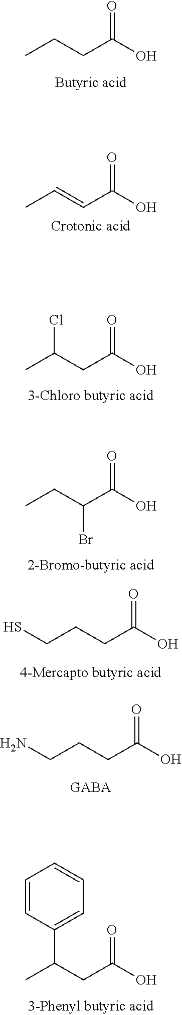

Butyrate and 4-Mercaptobutyrate (MBA) include the salts and acid forms and include but are not limited to: Butyric acid, crotonic acid, 3-Chloro butyric acid, 2-Bromo butyric acid, 4-Mercapto butyric acid, GABA, 3-Phenyl butyric acid and combinations thereof.

Butyrate (BA) and Barrier Preferred energy source for the colonic epithelium, oxidation of BA accounts for over 70% of O₂ consumption in the colon. 95% of BA is used by the colonocytes for energy. Large energy reserves are required for intestinal epithelial cells to rapidly polarize and to create strong adherens junction complexes.

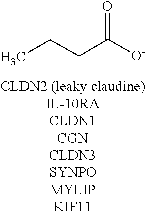

Butyrate regulates the barrier largely through stabilization of hypoxia inducible factors.

### Butyrate Stabilizes HIF Independent of Oxygen Consumption

Short-chain fatty acids (SCFAs) produced by the intestinal microbiota through anaerobic fermentation of undigested fiber have multiple roles within the human gut. Energy procurement depends on the metabolism of SCFAs, which also includes acetate, propionate, butyrate, and low amounts of valerate and hexanoate, through β-oxidation and contributes up to 15% of the host total daily caloric requirement. Total SCFAs concentrations can reach up to 150 mM in the colon. Butyrate is also a potent histone deacetylase (HDAC) inhibitor that regulates a plethora of intestinal genes. Together, butyrate fundamentally shapes the gut mucosa as both a transcriptional regulator and as an essential substrate for energy metabolism. Decreases in butyrate-producing bacteria and butyrate are key hallmarks of the dysbiosis seen in intestinal diseases.

The relationship between intestinal butyrate and hypoxia-inducible factor (HIF) lies at the intersection of metabolism and gene regulation. Due to the steep oxygen gradient that exists across the anoxic lumen and the highly oxygenated lamina propria, the intestinal mucosa exists in a state of particularly low pO₂ at baseline, a phenomenon termed “physiologic hypoxia.” Under such conditions, intestinal epithelial cells (IECs) that line the colon manifest stabilization of hypoxia-inducible factor (HIF). HIF is a master transcriptional regulator of numerous genes important to processes that include erythropoiesis, angiogenesis, energy metabolism, and inflammation. In normoxia, HIF-a subunits are degraded in an oxygen-dependent manner. When oxygen is limited, HIF-a is stabilized and forms a heterodimeric complex with HIF-1β in the nucleus to bind hypoxia responsive elements in the promoter region of hundreds of target genes. Three HIF-α isoforms (HIF-1α, HIF-2α, and HIF-3α) exist, but HIF-1α and HIF-2α are the best studied, and exhibit similar structures and function with unique and redundant targets. HIF-α stability is intimately controlled by oxygen levels, increasing slowly between atmospheric to 6% and then exponentially rising as oxygen levels approach 0.5%. The oxygen-sensitive nature of HIF proteins are reliant on HIF prolyl hydroxylases (HPHs), also known as prolyl hydroxylase domain (PHD) enzymes, which are primed to sense oxygen availability to provide exquisitely specific control of HIF stabilization, as any decrease in oxygen below atmospheric increases PHD enzymatic activity. PHDs belong to the superfamily of iron and 2-oxoglutarate (2-OG) dependent dioxygenases that utilize molecular oxygen to hydroxylate proline residues within the oxygen-dependent degradation domain (ODD) of HIF-α for recruitment of the von Hippel-Lindau tumor suppressor (pVHL), the recognition element of the E3 ubiquitin ligase that polyubiquitinates HIF-α for proteasomal degradation. Importantly, β-oxidation of butyrate for energy provision, through forming acetyl-CoA that enters the tricarboxylic acid (TCA) cycle to produce reducing equivalents that drive the electron transport chain to ultimately generate ATP, accounts for greater than 70% of cellular oxygen consumption in the distal colon, and this depletion of oxygen by butyrate is demonstrated to stabilize HIF.

The metabolism of butyrate stabilized HIF through a mechanism involving increased oxygen consumption. Utilizing the ATP synthase inhibitor oligomycin, it was revealed that oxygen consumption did not fully establish HIF stabilization. Residual HIF activity was evident in the presence of saturating concentrations of oligomycin.

Intestinal barrier dysfunction is directly linked to inflammatory bowel diseases. Gut barrier is regulated by a variety of microbial metabolites, including dietary fiber derived short chain fatty acids (e.g. butyrate). In disease, butyrate producing bacteria are lost and butyrate transporters are downregulated. Butyrate (BA) regulates barrier in part through stabilization of hypoxia inducible factor (HIF). HIF-deficient IECs exhibit major defects in mucosal barrier integrity. In normoxia, HIF is targeted for degradation catalyzed by prolyl hydroxylase enzymes (PHD1-3), mainly PHD2.

BA is a direct and non-competitive inhibitor of PHD2. BA inhibits PHD activity. PHD reaction hindered by BA as observed by accumulation of substrate 2-oxoglutarate (2-OG). Dose dependent inhibition of PHD2 monitored by succinate concentration (DMOG/IOX2=PHD inhibitors). BA non-competitively binds to PHD2. WaterLOGSY ID NMR to determine BA binding to recombinant PHD2. IC-50 values for BA and DMOG in increasing concentration of 2-OG are shown in the figures. >95% of native BA is used by colonocytes for energy procurement.

The present disclosure teaches “well tolerated” small molecule inhibitors of PHDs to stabilize HIF in the treatment numerous disorders. Analogues structurally related to butyrate more specifically stabilize HIF with a longer biological half-life. A screen of structural butyrate analogs identified 4-Mercaptobutyrate (SHBA) as a potent HIF stabilizer in cultured epithelial cells.

SHBA stabilizes HIF. BA derivatives were investigated in CaCo2 cells (5 mM, 6 h), identifying SHBA as a PHD inhibitor (Veh=HBSS+). (B) Western blot of HIF protein levels in treated CaCo2 cells (5 mM, 6 h). Data presented as mean±S.E.M, **p<0.01. Time course studies of SHBA-HIF stabilization. Western blots of HIF1a and HIF target (BNIP3) protein over time with 5 mM BA and SHBA treatments in Caco2 (A) and HIF1a in T84 (B). Mesoscale assay of HIF1a protein over time in Caco2 cells treated with 5 mM SHBA. Data are presented as mean±S.E.M, *p<0.05, **p<0.01, ***p<0.001.

SHBA transactivates HIF target genes. (A) Exposure of T84 cells to Veh, BA or SHBA (5 mM, 16 h) induced HIF target gene expression. (B) Influence of SHBA (5 mM, 16 h) on the expression of HIF targets in T84 cells lacking HIF1b (HIF1b KD) relative to control (sh Ctl). Presented as mean±S.E.M, *p<0.05, **p<0.01. Barrier formation response to BA and SHBA. Caco2bbe cell barrier development in Veh, BA or SHBA treated cells (5 mM); n=3. Data presented as mean±S.D. ****p<0.0001.

C57BL/6 mice age 8-12 weeks injected IP with PBS (200 μL), PHD inhibitor DMOG (117 mg/kg), BA (73 mg/kg), and SHBA (80 mg/kg). At 24 h, mice were sacrificed followed by exsanguination, extraction of right kidney and distal colon for analysis of HIF stabilization and HIF target gene expression. SHBA induces HIF regulated targets in vivo. Erythropoietin (EPO) is regulated by HIF stabilization. (A) EPO protein in blood serum was assessed by mesoscale assay (n=6-8). (B) qPCR mRNA expression of HIF target mGLUT1 in colon tissue (n=3-4). Data are presented as mean±S.E.M of two independent experiments, *p<0.05, **p<0.01.

SHBA stabilizes HIF for a longer period of time compared to BA in cultured epithelial cells. Confirmed induction of classic HIF target genes through HIF stabilization. Increased and sustained barrier formation in vitro when treated with SHBA. BA analogs induce of HIF targets in vivo. SHBA (and other) BA analogues serve as chemical templates for therapeutics in IBD.

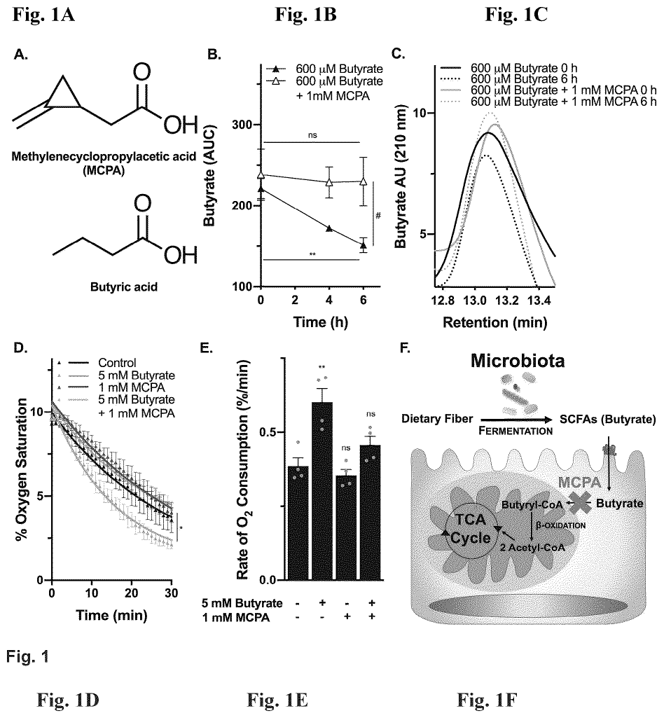

Increased β-oxidation and consequent oxygen consumption by butyrate metabolism were evaluated utilizing methylenecyclopropylacetic acid (MCPA) to irreversibly inhibit SCFA acyl-CoA dehydrogenases, most potently and specifically butyryl-CoA dehydrogenase to block butyrate β-oxidation (FIG. 1A). MCPA has been shown to irreversibly inhibit the metabolism of butyrate and reduce acetyl-CoA levels by 70-90% in rat hepatocytes. MCPA reduced butyrate metabolism in T84 cells (FIG. 1B-C) and eliminated the increase in oxygen consumption associated with butyrate (FIG. 1D-E). As depicted in FIG. 1F, MCPA inhibits-oxidation of butyrate, preventing butyrate-derived acetyl-CoA production and therefore butyrate-induced TCA cycle flux and oxygen consumption through oxidative phosphorylation/aerobic respiration.

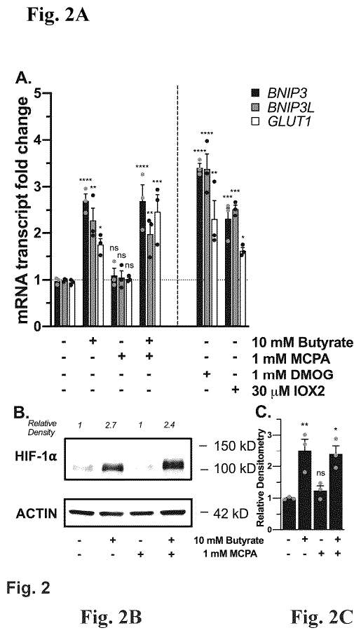

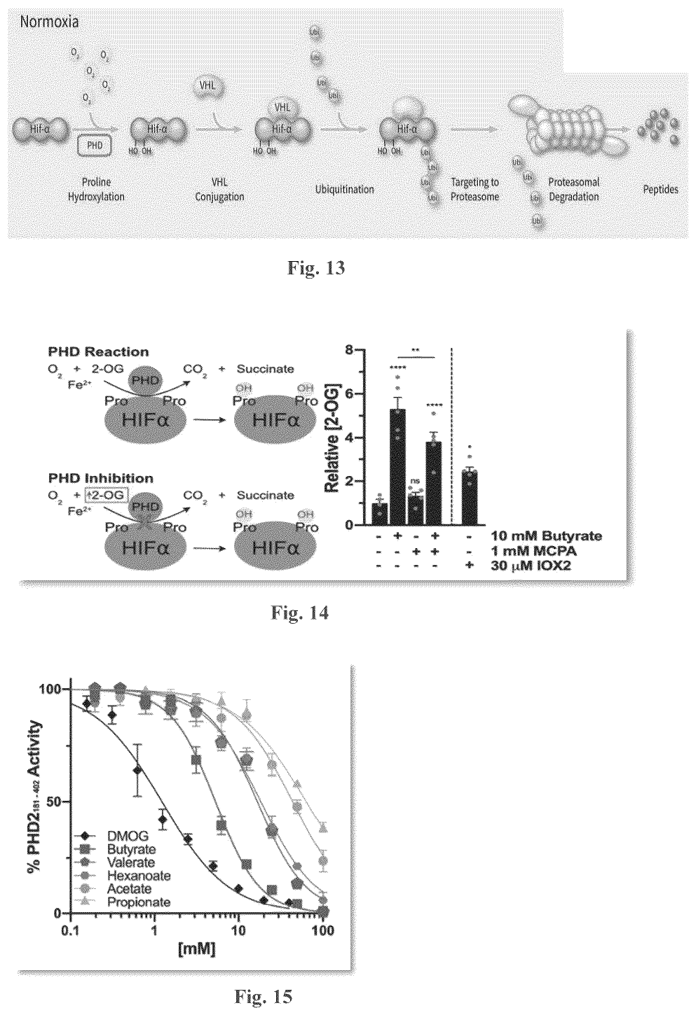

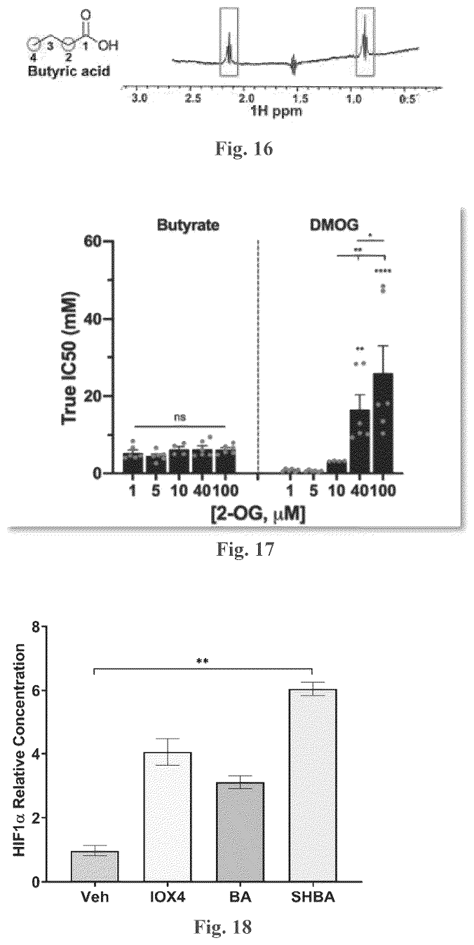

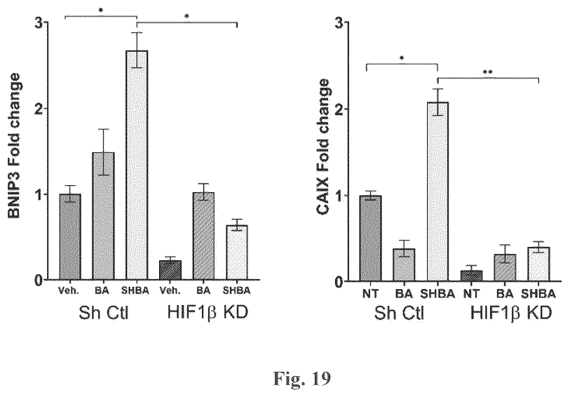

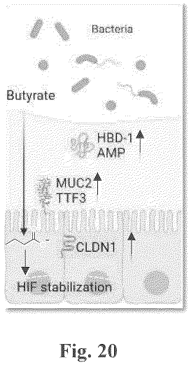

In T84 adenocarcinoma model IECs the ability of butyrate to stabilize HIF through the induction of HIF-1α target genes BNIP3, BNIP3L, and GLUT1 (FIG. 2A), similar to dimethyloxalylglycine (DMOG), a 2-OG analogue with broad-spectrum inhibition of PHDs, and IOX2, a more PHD2 specific inhibitor. HIF-1α protein levels were also increased with butyrate (FIG. 2B-C). In the presence of MCPA, butyrate still stabilized HIF (FIG. 2A-D), thus, butyrate does not stabilize HIF solely through limiting oxygen availability. We found similar HIF stabilization in A549 lung adenocarcinoma epithelial cells and HMEC-1 human dermal microvascular endothelial cells seen by HIF-1α target gene induction and increased HIF-1α protein levels (Supplemental FIGS. 11-13), suggesting a more universal response beyond IECs.

### Butyrate Increases 2-OG Similar to PHD Inhibition

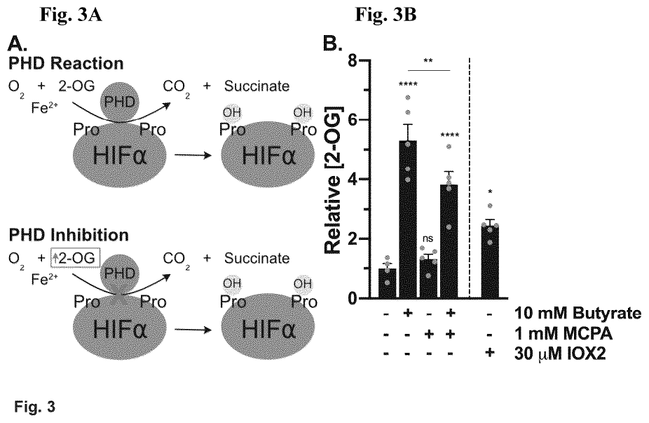

The relationship between butyrate and HIF, were examined whether butyrate influences PHD activity by monitoring 2-OG levels. PHD2 inhibition directly leads to 2-OG accumulation, as PHD2 decarboxylates 2-OG at a high rate of ˜200 pmol/min/g of tissue, with 1 mole of PHD2 estimated to decarboxylate 45 moles of 2-OG in 1 min. In this, we considered such 2-OG accumulation as a metabolic biomarker of PHD inhibition (FIG. 3A). Butyrate significantly increased 2-OG levels in T84 IECs compared to control after 3 h, as did the PHD inhibitor IOX2, albeit to lesser extent than butyrate at this time point (FIG. 3B). In the presence of MCPA and butyrate, which eliminated the β-oxidation of butyrate, 2-OG levels were also significantly increased, expectedly to a significantly lesser level compared to butyrate alone (FIG. 3B). MCPA, through eliminating β-oxidation of butyrate, not only stops the increase in oxygen consumption, but also prevents the increased TCA cycle production of metabolites such as 2-OG (FIG. 1F). Because 2-OG is also a TCA cycle metabolite, the additional increase in 2-OG with butyrate compared to IOX2 and butyrate with MCPA represents the 2-OG produced from the TCA cycle because of butyrate β-oxidation. Butyrate treatment over 4 days has been shown to significantly increase acetyl-CoA and 2-OG levels in other model IECs. While butyrate metabolism contributes to increased 2-OG, butyrate additionally increases 2-OG levels in a manner independent of β-oxidation that is possible through direct PHD inhibition.

### Butyrate Directly Inhibits Recombinant PHD2₁₈₁₋₄₀₂

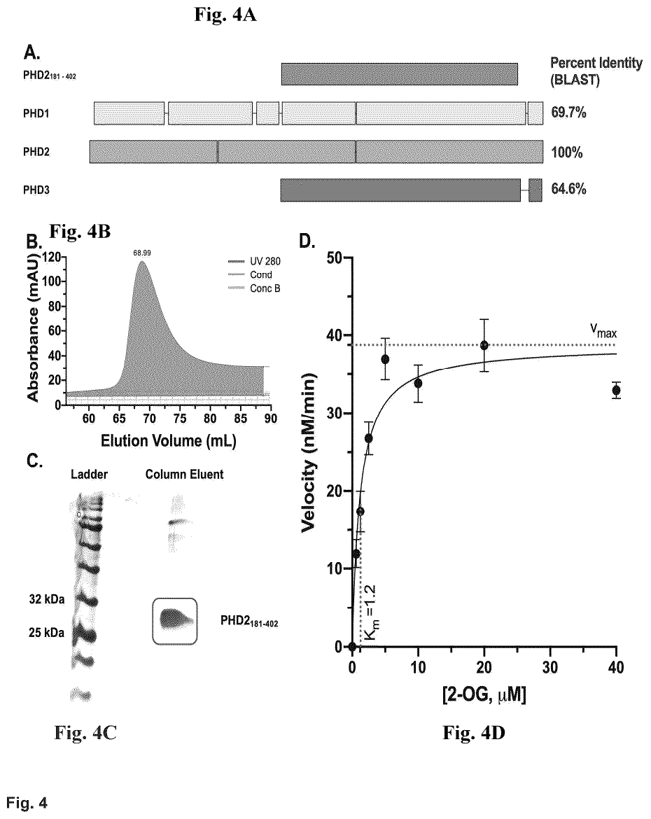

To pinpoint if butyrate inhibits PHDs directly, we expressed recombinant PHD derived from the human PHD2 sequence. There are three human PHDs: PHD1, PHD2, and PHD3, with PHD2 being the most abundant and expressed in the majority of tissues, the most important regulator of HIF, particularly HIF-1α, and the only PHD that is embryonically lethal when deleted. PHD1 is exclusively localized to the nucleus, whereas PHD2 is mainly cytoplasmic, and PHD3 is both. While PHD2₁₈₁₋₄₂₆ is the commonly utilized catalytic domain, we expressed PHD2₁₈₁₋₄₀₂, which is similar in activity. Sequence comparisons and modeling studies indicate that the PHD2 active site is highly conserved among the PHDs. We posit that the results garnered from the recombinant PHD2 are likely applicable to all PHDs, as PHD2₁₈₁₋₄₀₂ spans the conserved active site while excluding specificity determining regions in the N-terminal domains (FIG. 4A). While PHD2₁₈₁₋₄₀₂ does contain the non-homologous β2β3 loop within the catalytic domain that determines the preference of PHD3 for the C-terminal ODD (CODD) of HIF-1α, the HIF-1α peptide utilized in our assays only contains the CODD and mitigates such specificity differences. Recombinant PHD2 was purified prior to use on size exclusion column Superdex 75, and the expected size of 28.9 kDa was verified on SDS gel electrophoresis (FIGS. 4B-C).

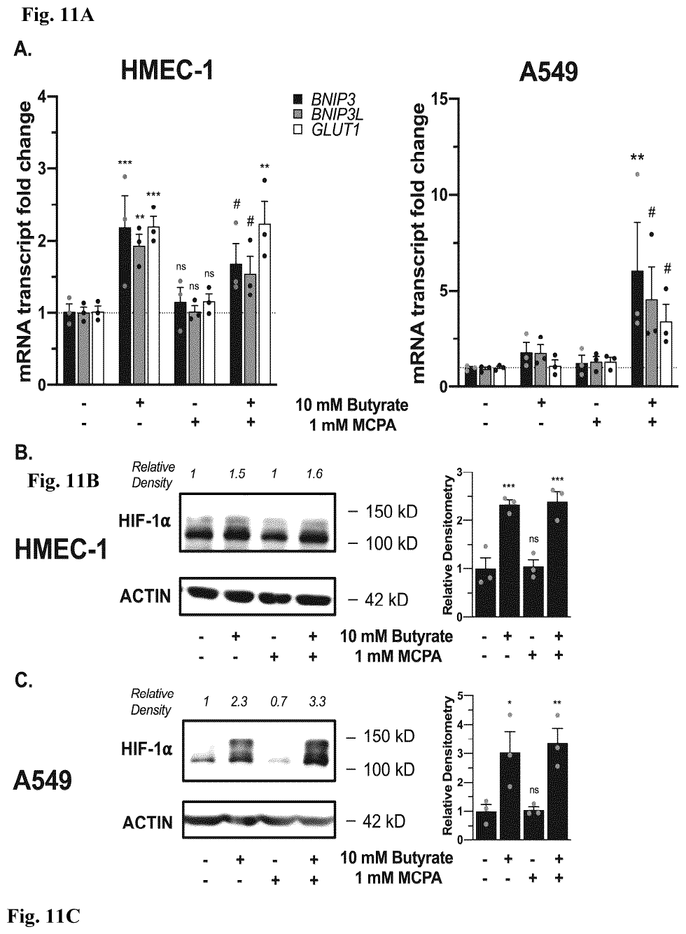

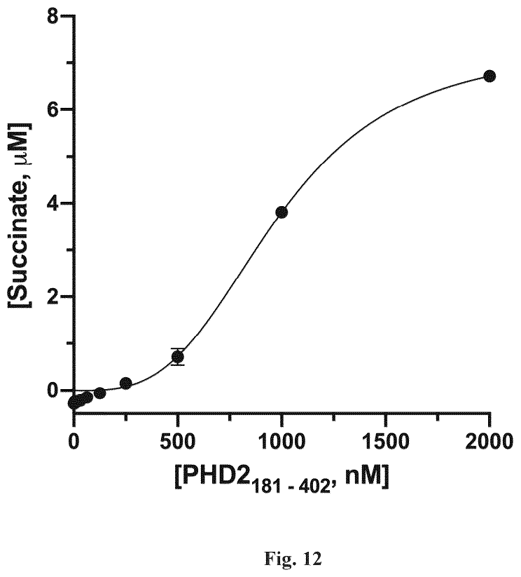

We next evaluated the activity of PHD2₁₈₁₋₄₀₂ using a bioluminescent succinate detection assay as described by Alves et al. PHD2₁₈₁₋₄₀₂ was confirmed to be catalytically active with an optimal protein concentration for the assay of 1 μM (Supplemental FIG. 12), in agreement with literature and allowing the reaction to remain within the limits of detection for the assay. The Km of 2-OG for PHD2₁₈₁₋₄₀₂ was determined as 1.2 μM (FIG. 4D), in agreement with literature.

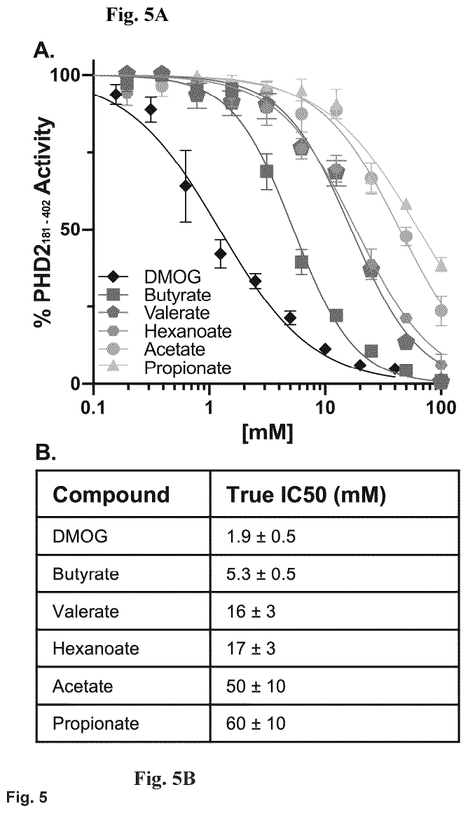

Butyrate most potently inhibited PHD2₁₈₁₋₄₀₂ compared to all other tested SCFAs, with a true half maximal inhibitory concentration (IC50) of 5.3+0.5 mM (FIGS. 5A-B). Shorter SCFAs acetate and propionate were ˜10-fold less effective at inhibiting PHD2₁₈₁₋₄₀₂, and longer SCFAs valerate and hexanoate were ˜3-fold less effective. For the inhibition studies, we calculated true IC50s from measured IC50s with the equation derived by Wu et al. describing the relationship between the measured IC50 of an inhibitor and the percentage of substrate conversion, as Michaelis-Menten kinetics require substrate conversion to be below 10%, which would produce signals difficult to detect with our assay. As a positive control in support of the assay, we determined the true IC50 for DMOG to be 1.9±0.5 mM, within the range of reported IC50s between 2.89 μM to 4 mM and is broad due to the inherent dependency of IC50s on assay conditions that varies across studies. Thus, butyrate is comparable to DMOG as a PHD inhibitor.

Butyrate binds directly and specifically to PHD2₁₈₁₋₄₀₂

Next, we established whether butyrate binds directly to PHD2₁₈₁₋₄₀₂ using 1D WaterLOGSY NMR. This method enables sensitive and robust detection of binding (dissociation constants as weak as μM to low mM) based on the magnetization transfer between water molecules, proteins, and ligands of interest in close proximity via dipolar proton-proton cross-relaxation (nuclear Overhauser effect, NOE). Protein-ligand complexes exhibit an opposing NOE with water, resulting in a positive WaterLOGSY signal, while molecules that do not bind to protein exhibit a weak and same NOE with water, resulting in a negative WaterLOGSY signal. Binding and non-binding ligands can be distinguished in a WaterLOGSY spectrum via their opposite signs in relation to water for their corresponding peaks.

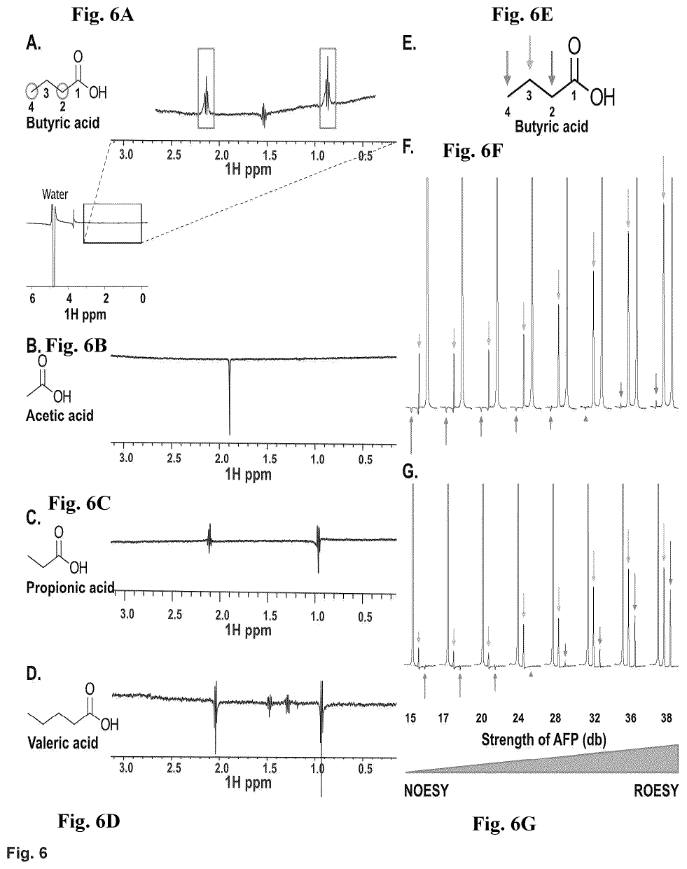

The combination of PHD2₁₈₁₋₄₀₂ and butyrate revealed two inverted proton peaks at 2.02 and 0.75 ppm compared to the water signal and demonstrate a clear positive signal, indicative of binding (FIG. 6A). By contrast, we did not observe binding of other SCFAs under similar conditions, (FIG. 6B-D), suggesting that SCFA binding to PHD is specific for butyrate. We further validated the specific butyrate protons involved in the butyrate-PHD2₁₈₁₋₄₀₂ interaction through 1D AFP-NOESY NMR (FIG. 6E), in which a linear combination of two different proton-proton cross-relaxation rates (NOEs and rotating-frame Overhauser effects, ROEs) are measured during adiabatic fast passage (AFP). This technique allows for mapping of the ligand pharmacophore by monitoring the response of individual protons to an increase of ROEs contribution to the overall cross relaxation rate. The protons resonating at 2.02 (FIG. 6F) and 0.75 ppm (FIG. 6G) experienced “spin diffusion” modification, indicative of deeper imbedding in the binding pocket, while the proton resonating at 1.41 ppm did not exhibit such a trend (FIGS. 6F-G). These experiments together established that the protons bound to carbons C2 (2.02 ppm) and C4 (0.75 ppm) of butyrate directly interact with PHD2₁₈₁₋₄₀₂, while the protons attached to C3 (1.41 ppm) appear to be more solvent exposed. In agreement with these results, any C2 or C4 modification resulted in significant decreases in inhibition by increasing IC50s, whereas modifications to C3 decreased inhibition to a lesser extent (Table 1). Together, these results confer butyrate as a highly specific PHD 181-402 inhibitor compared to other SCFAs.

TABLE 1
True IC50 values of butyrate-derived compounds with modifications on
different carbons. Modifications to C2 and C4 of butyrate significantly decrease inhibition of
compounds for PHD2181-402, while modifications to C3 impact inhibition to a lesser extent
(n = 4, error: SEM)

| Compound | Structure | True IC50 (mM) |
|---|---|---|
| Butyrate | 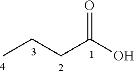 | 5.3 ± 0.5 |
| 2-Ethylbutyrate | 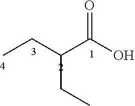 | 77 ± 6 |
| 2-Methylbutyrate | 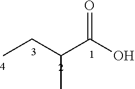 | 77.2 ± 0.3 |
| 3-Hydroxybutyrate | 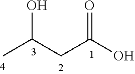 | 14 ± 1 |
| 4-Acetylbutyrate | 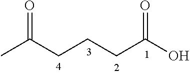 | 220 ± 50 |

Butyrate noncompetitively inhibits PHD2₁₈₁₋₄₀₂ with a Kᵢ of 5.3 mM

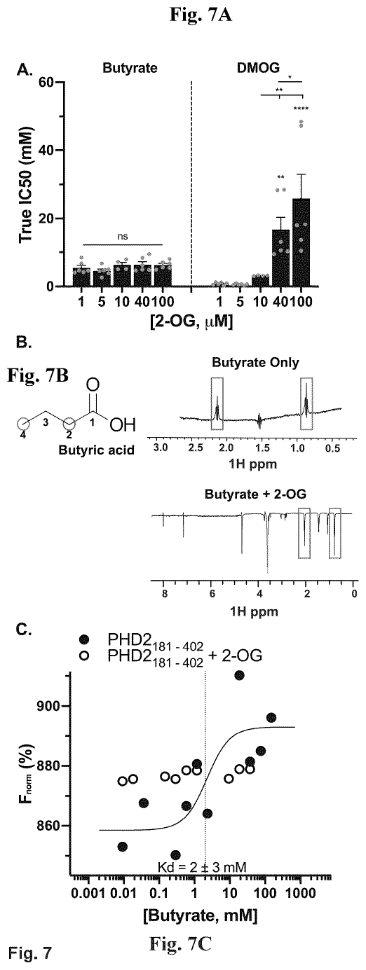

By titrating different concentrations of substrate 2-OG into the inhibition assay and assessing if IC50s increased, we found the mode of butyrate inhibition to be either noncompetitive or uncompetitive. IC50s for butyrate did not change with increasing 2-OG (FIG. 7A), whereas IC50s for DMOG increased significantly with increasing 2-OG, in agreement with DMOG as a known competitive 2-OG analogue. Uncompetitive inhibitors only bind to the enzyme substrate complex, whereas noncompetitive inhibitors can bind to enzyme alone or enzyme substrate complex since the inhibitor has a unique binding site distinct from the substrate binding site. We found that butyrate could not bind to the 2-OG and PHD2₁₈₁₋₄₀₂ complex, as the inverted peaks at 2.02 and 0.75 ppm were lost on WaterLOGSY 1D NMR (FIG. 7B), eliminating uncompetitive inhibition as a binding mechanism. We further confirmed butyrate binding to PHD2₁₈₁₋₄₀₂ using microscale thermophoresis (MST), which revealed a dissociation constant (KD) for butyrate of 2±3 mM (FIG. 7C), comparable to our true IC50 for butyrate. Again, in the presence of 2-OG, MST revealed no butyrate binding to PHD2₁₈₁₋₄₀₂ (FIG. 7C). In the context of these assays, the lack of butyrate binding to PHD2₁₈₁₋₄₀₂ in the presence of 2-OG does not represent competitive inhibition, as we already ruled out competitive inhibition with the data shown in FIG. 7A, suggesting that butyrate binds PHD2₁₈₁₋₄₀₂ elsewhere of the 2-OG binding site. Allosteric regulation could result in the formation of the 2-OG and PHD2₁₈₁₋₄₀₂ complex causing a conformational change in PHD2₁₈₁₋₄₀₂ that precludes butyrate binding, similar to the way a noncompetitive inhibitor like butyrate could bind to PHD2₁₈₁₋₄₀₂ and cause a conformational change that precludes substrates like 2-OG from binding. Thus, our findings indicate that butyrate functions as a noncompetitive PHD inhibitor with a unique binding site.

IC50s depend on exact experimental conditions, making direct comparisons and global applications difficult. We utilized the IC50 to Kᵢ conversion equation detailed by Cer et al. to determine the intrinsic inhibitory constant (Kᵢ) of butyrate for PHD2₁₈₁₋₄₀₂ that is independent of experimental variables. We calculated the noncompetitive Kᵢ of butyrate for PHD2₁₈₁₋₄₀₂ to be 5.3 mM, a physiologically relevant concentration for butyrate in vivo.

### Microbiota-Derived Butyrate is Essential to Stabilizing Colonic HIF

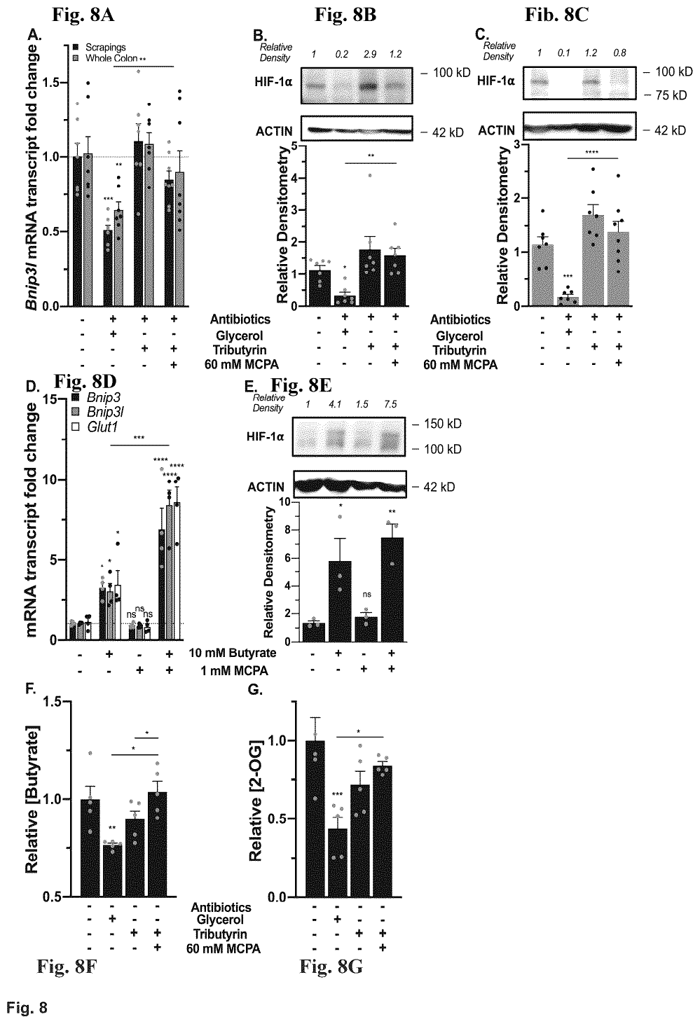

We next confirmed that butyrate functions as a PHD inhibitor in vivo through antibiotic-mediated depletion of the gut microbiota and SCFAs in mice. This approach has been previously published and validated by our group and was shown to deplete all SCFA by >90%. We administered tributyrin (200 μL), a prodrug of butyrate composed of a glycerol backbone with three butyrate moieties, to SCFA-depleted mice via oral gavage every day for three days with glycerol as a control. Additionally, some mice given tributyrin were subjected with a rectal gavage of 60 mM MCPA to inhibit butyrate metabolism 4 h prior to sacrifice. Previous studies have found that oral gavage with tributyrin elevates plasma butyrate concentrations to >1 mM 1 h after dosing in mice. 60 mM MCPA was chosen as a well-tolerated dose that inhibited β-oxidation in mice and rats. This analysis revealed in both whole colon tissue and intestinal epithelial scrapings from these mice that antibiotic treatment decreased HIF stabilization as HIF-1α target gene Bnip31 was significantly decreased that was normalized by both tributyrin supplementation with or without MCPA (FIG. 8A). HIF-la protein was also diminished in epithelial scrapings (FIG. 8B) and whole colon tissue (FIG. 8C) with antibiotic treatment, which was also rescued with tributyrin supplementation with or without MCPA. Additionally, we confirmed in differentiated murine enteroids that butyrate with or without MCPA treatment induced expression of HIF-1α target genes (FIG. 8D), as well as increased HIF-1α protein expression after 4 h (FIG. 8E). These results, in conjunction with the colon epithelial scrapings, confirm that PHD inhibition does occur in epithelial cells in a similar manner as our in vitro experiments in T84 IECs revealed. The stabilization of HIF-1α in whole colon tissue also is in agreement with our findings in HMEC-1 and A549 cells that PHD inhibition by butyrate expands to beyond just IECs alone. We confirmed that colon tissue butyrate levels were significantly decreased with antibiotic treatment (glycerol control) and were then rescued by tributyrin with or without MCPA treatment (FIG. 8F). As MCPA is a butyrate metabolism inhibitor, the MCPA rectal gavage in mice given tributyrin significantly increased the level of butyrate in the colon tissue compared to just tributyrin alone. Lastly, antibiotic treatment depleted 2-OG levels in the mice colon tissues that was then recovered by tributyrin treatment with or without MCPA (FIG. 8G). Our results teach that microbiota-derived butyrate also stabilizes HIF through direct PHD inhibition, as indicated by HIF stabilization, induction of HIF-1α mRNA targets, and a 2-OG increase even during MCPA inhibition of butyrate metabolism (TCA cycle) and accompanying oxygen consumption.

To confirm these results, we reconstituted butyrate in a second manner in SCFA-depleted mice. These mice were rectally administered 100 mM butyrate with or without 10 mM MCPA or 1.5 mM of the PHD2 inhibitor IOX2 acutely for 1 h. Daily 100 mM butyrate enemas over weeks have been used therapeutically in both rat and mice animal studies and human clinical trials. Doses for MCPA and IOX2 were selected to be optimized for their influences as well as be tolerated in the rapid 1 h time point, which was selected as Olenchock et al demonstrated that 2-OG levels increased in liver tissues after pharmacological PHD inhibition to a peak within 10 minutes and decreased to nonsignificant levels after 4 h. Furthermore, in addition to the possibility that butyrate could inhibit PHDs and elicit measurable responses in a rapid manner, we targeted 1 h to decrease the likelihood of seeing metabolic shifts due to butyrate metabolism in addition to PHD inhibition alone, similar to our T84 in vitro studies.

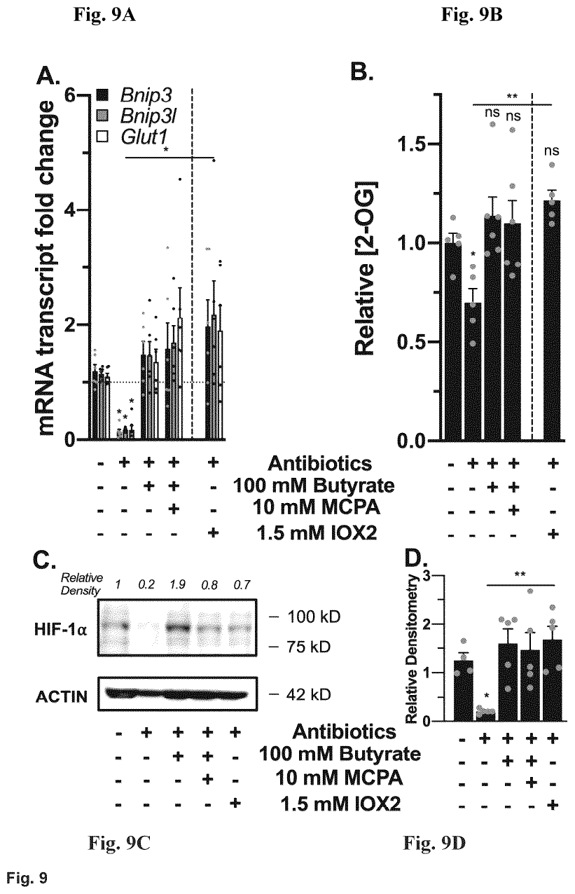

Antibiotics significantly decreased mRNA expression of HIF-1α targets Bnip3, Bnip3l, and Glu1 that were rescued with butyrate, butyrate with MCPA, and IOX2 (FIG. 9A). Similarly, using colon tissue 2-OG levels as a biomarker for PHD inhibition, we observed a significant decrease in 2-OG levels with antibiotics that were rescued with both butyrate and IOX-2 (FIG. 9B). Lastly, western blot analysis revealed the loss of HIF-1α protein stabilization with antibiotics that returned with all treatments (FIG. 9C-D), showing that butyrate functions as a direct PHD inhibitor in vivo.

Microbiota-derived butyrate is critical to maintaining intestinal homeostasis, and dysbiosis of the microbiota in disease states commonly diminishes butyrate levels through decreasing butyrate-producing bacteria, notably in inflammatory bowel diseases (IBD). IBD colonocytes do not effectively transport nor metabolize butyrate, and germ-free mice lacking in butyrate show diminished oxidative metabolism and energy deficiency. Butyrate inhibition of PHDs stabilizes HIF-1α, which regulates many critical gut homeostasis genes including claudin 1 (CLDN1), an essential tight junction protein, mucin 2 (MUC2), the major component of the mucus layer, and human beta defensin-1 (DEFB1), an antimicrobial peptide.

IECs exist in a state of perpetual low oxygen tension, a phenomenon termed “physiologic hypoxia.” This is partially due to proximity to the anaerobic colonic lumen, which establishes a radial oxygen gradient with the intestinal epithelium residing at a pO₂ of less than 10 mmHg or ˜1% oxygen to ˜5-10% in the vascularized submucosa and muscle layers, but also results from the consumption of oxygen stemming from the metabolism of microbiota-derived butyrate. Germ-free and antibiotic-treated mice show diminished physiologic hypoxia, secondary to lacking intact gut microbiotas and butyrate. In this the colonic mucosa contributes to an environment with low oxygen levels, in which HIF is stabilized at baseline. We teach that butyrate directly inhibits PHDs to stabilize HIF gives precise regulation of HIF by the microbiota. As the ETC can function at near anoxia and only becomes limited by intracellular oxygen when levels reach below 0.3%, temporally, butyrate binding to and inhibiting PHDs to stabilize HIF would occur before oxygen becomes limiting from butyrate metabolism. Not only does additional HIF stabilization beyond the oxygen-regulated baseline confer homeostatic benefit, but this rapid stabilization of HIF could also play a major role in priming the colon tissue towards butyrate metabolism in the already oxygen-deprived environment. HIF-1α has been to show to upregulate pyruvate dehydrogenase kinases (PDKs), PDK1 and PDK3, which inactivate the pyruvate dehydrogenase complex (PDC) by inhibiting pyruvate dehydrogenase (PDH) to prevent glucose-derived pyruvate conversion into acetyl-CoA and entering the TCA cycle, and thus shifts the production of acetyl-CoA to be from β-oxidation of butyrate. Butyrate has also been shown to strongly induce PDK1-4 through HDAC inhibition. Ultimately, in the unique environment of the colon, butyrate directly and indirectly through HIF-1α stabilization induces PDKs to shift acetyl-CoA production from glycolysis to butyrate oxidation, cementing the importance and specificity of butyrate to the colon. Microbiota-derived butyrate as an essential component of intestinal homeostasis.

Butyrate inhibition of PHDs also influences intestinal homeostasis through regulating metabolite levels, specifically 2-OG. Increased 2-OG from PHD inhibition drives production of kynurenine, which protects against cardiac ischemia. Kynurenine, as a tryptophan derivative, promotes intestinal wound healing and alleviates murine colitis, but can be a downstream beneficial influence of accumulated 2-OG from butyrate PHD inhibition. 2-OG inhibits colorectal carcinogenesis, and 2-OG supplementation downregulates inflammatory cytokines IL-6, IL-22, TNF-α, and IL-1β along with decreasing opportunistic pathogens and increasing mutualistic bacteria including the butyrate-producing class Clostridia in the colon. Again, due to the temporal differences in 2-OG levels due to the route of production, whether rapidly from direct inhibition of PHDs or more delayed from β-oxidation, butyrate exhibits meticulous control over the intestinal mucosa. As observed with DMOG and IOX2 treatment in T84 cells, PHD inhibition alone raised 2-OG to a certain level, whereas butyrate demonstrated the capacity to further elevate 2-OG due to both metabolism and PHD inhibition. This highlights an important therapeutic consideration in that PHD inhibition and the contribution of butyrate β-oxidation to the TCA cycle and 2-OG production are co-foundational components of gut homeostasis and may both be necessary for wound healing.

The microbiota produces butyrate to reach levels of 15-25 mM in the colon, and due to differential apical and basolateral affinities of the SCFA-HCO3 exchange transporters, >95% is absorbed and sequestered to the colonic mucosa for signaling and metabolism, with only low micromolar concentration (<2% of colon-derived) found in portal blood, and the remaining secreted in feces. However, most acetate and propionate are delivered to and utilized by the liver, with acetate being the only SCFA to enter peripheral circulation at high enough concentrations to additionally influence heart, adipose, kidney, and muscle activity. Thus, it is likely that butyrate selectively inhibits PHDs in the colon, while other SCFAs do not reach sufficient levels for such inhibition. It is also notable that such selectivity of butyrate in the colon is not unexpected, as butyrate also most potently inhibits HDACs, while propionate, valerate, and hexanoate exhibit lesser degrees of HDAC inhibition, and acetate shows none.

Butyrate, and derivatives disclosed herein, are a well-tolerated, endogenous metabolites, with few deleterious side effects, especially as a noncompetitive inhibitor. It and its derivatives are small-molecule inhibitors of PHDs to stabilize HIF for the treatment of numerous disorders including intestinal disease, novel in that previous PHD inhibitors have only focused on targeting the active site. Other iron-chelator classes of inhibitors are designated “noncompetitive” but function to limit iron availability in the active site. Other endogenous inhibitors (e.g. TCA cycle succinate) are considered competitive inhibitors. This disclosure teaches a truly noncompetitive inhibitor with a unique binding site.

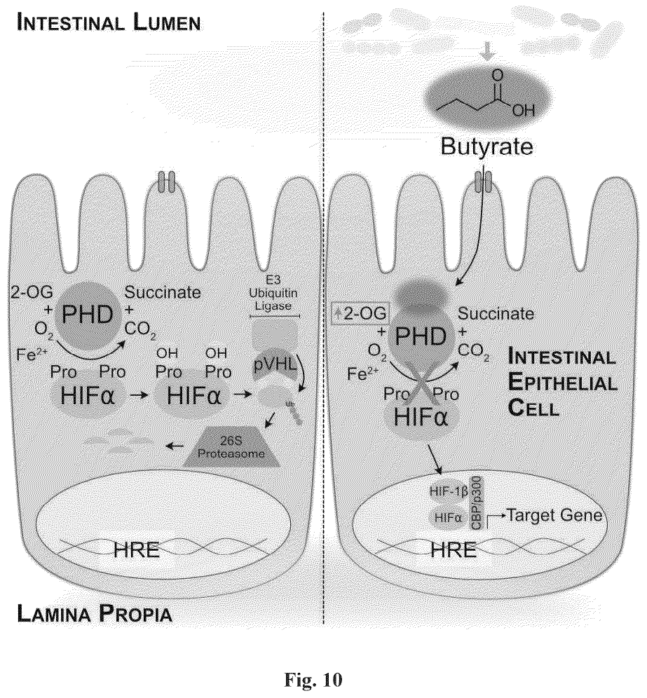

Co-evolution of mammals and the gut microbiota has created a complex system in which microbiota-derived butyrate is made available to inhibit PHDs at a specific site and in a tissue-specific manner. However, the introduction of butyrate to other systems could be beneficial in inhibiting PHDs and stabilizing HIF. Our in vitro work indicates that butyrate can stabilize HIF in non-intestinal cells and suggests that while organs outside of the colon do not normally experience high concentrations of butyrate, the SCFA may still significantly influence their function. The varied magnitude of HIF-1α gene target responses to butyrate and butyrate with MCPA in A549 cells compared to T84 cells confirm that HIF differentially regulates genes across organ systems, and that butyrate is uniquely metabolized by each organ and exerts distinct influences. Our in vivo work also demonstrates that PHD inhibition by butyrate extends beyond just IECs. Overall, our work here demonstrates that microbiota-derived butyrate binds and noncompetitively inhibits PHDs (FIG. 10). Such inhibition stabilizes HIF and increases 2-OG, all of which contribute to gut homeostasis. Butyrate as a noncompetitive PHD inhibitor not only provides insight into PHD/HIF therapeutics but also highlights the intricate mutualistic symbiosis that exists between mammals and the microbiota.

### EXAMPLES

It will be apparent to those skilled in the art that various modifications and variations can be made in the methods and compositions of the present invention without departing from the spirit or scope of the invention

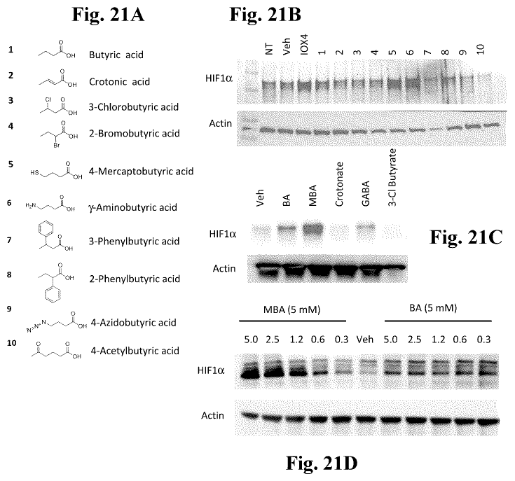

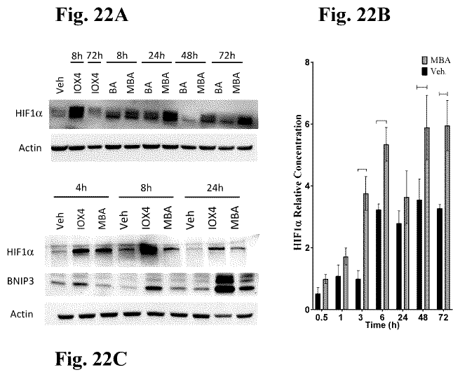

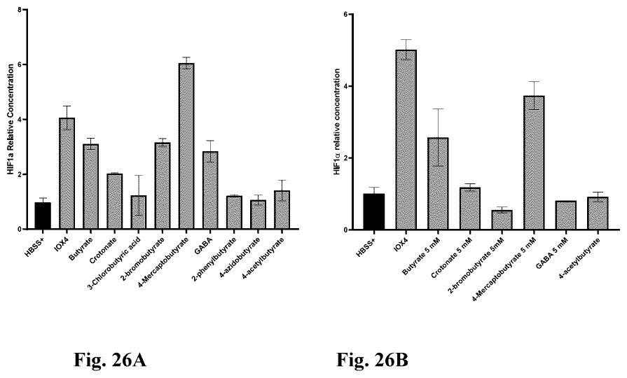

Endogenous and non-endogenous butyrate derivatives with specifically chosen functional groups at different positions within the small molecule and assess their effects on HIF stabilization (FIG. 21A). To study a small library of butyrate derivatives we employed an electrochemiluminescence based assay to quantify HIF1α levels using the Meso Scale Discovery (HIF1α MSD assay, http://www.mesoscale.com) technology. Similar to the sandwich enzyme-linked immunosorbent assay (ELISA), this technology captures and detects HIF1α protein using two specific antibodies but utilizes electrochemiluminescence-based signal generation and detection. Using lysates from intestinal epithelial cells (IECs) caco-2 and T84 exposed to a physiological relevant concentration of 5 mM butyrate derivatives for 4h, this assay was employed to determine HIF1α protein concentration. A bona-fide and potent PHD2 inhibitor (IOX4) was used to stabilize HIF1α as a positive control. Various butyrate derivatives increased HIF1α relative concentration in both cell lines compared to low signals detected in untreated lysates. One of the butyrate derivatives exceeded HIF stabilization in caco-2 compared to the potent IOX4 PHD2 inhibitor (albeit at different concentrations) identified as 4-mercapto butyrate (MBA) (FIG. 26). This result was confirmed by immunoblotting for HIF1α, and as observed in FIG. 1B and C, MBA stabilizes HIF1α at least as much as IOX4 (10 μM) in caco-2 and exceeding the capabilities of butyrate at an equimolar concentration in both cell lines. We decided to focus on this interesting butyrate derivative for further studies. A dose response study between MBA and BA demonstrated that MBA can stabilize HIF at the low millimolar range, where BA quickly becomes ineffective, demonstrating an important characteristic that could be clinically relevant (FIG. 21D). Based on this result and for a fair comparison, a 5 mM concentration of MBA and BA was used in further studies.

### 4-Mercapto Butyrate Extends HIF1α Protein Life Compared to Native Butyrate

To elucidate the effectiveness of MBA to stabilize HIF1α over time and possibly shine light on how quickly it can be metabolized in-vitro, time-course studies where performed between BA and MBA, IOX4 was used as a positive control. As observed in FIG. 22A, HIF1α is no longer stabilized at 48h when T84 cells are exposed to butyrate, possibly due to effective metabolism. In contrast, the effect of MBA on HIF stabilization is observed clearly up to 72h. Interestingly, IOX4 is no longer effective at the 72h timepoint. Employing the HIF1α MSD assay, we screened for various timepoints in caco-2 cell lysates exposed to MBA (5 mM). As observed in FIG. 22B, HIF1α is rapidly stabilized (3h) and its relative concentration steadily increases over time compared to untreated up to 72h, thus supporting its prolonged stabilization effect. BNIP3 is involved in cell survival and is a known HIF1α target that is induced through chemical stabilization of HIF or hypoxia. BNIP3 promoter comprises a functional hypoxia response element (HRE) and can be activated by HIF1α.[5] Parallel to our HIF stabilization time course studies, we wanted to assess the transcriptional activity of the stabilized protein over time. BNIP3 Immunoblotting of c2bbe cell lysates exposed to IOX4 and MBA over various timepoints supported active HIF1α transcription, more specifically at longer timepoints (FIG. 2C).

HIF Dependent Transcription of Target Genes after Exposure to 4-Mercapto Butyrate

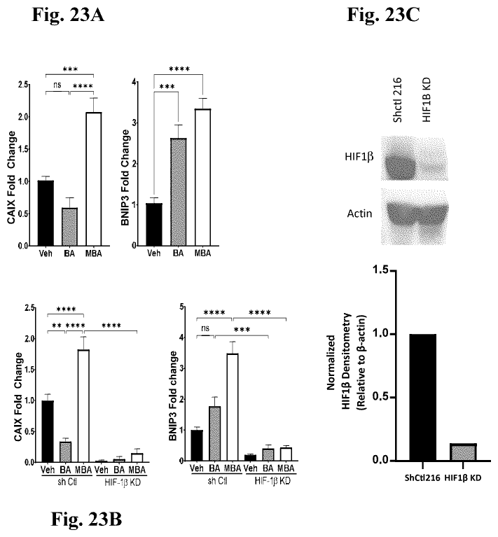

Following these findings, we then explored if the MBA-induced stabilization of HIF was reflected in the transcriptional regulation of target genes. We utilized real-time PCR to study the influence of MBA mediated HIF stabilization in the induction of established HIF targets, BNIP3 (cell survival) and CAIX (pH regulation). BA was used both for comparison and as a positive control in these experiments since it has been previously established to induce HIF targets significantly.[6] As shown in FIG. 23A, intestinal epithelia exposed to 5 mM MBA or BA for 0 and 16h resulted in significant induction of these target genes, however, in both cases MBA was more potent than BA.

Furthermore, to confirm MBA-induced HIF transcriptional activity, we quantified the induction of BNIP3 and CAIX by BA or MBA (5 mM, 16h) in lentiviral shRNA-mediated knockdown of HIF1β relative to non-targeting shRNA controls. As observed in FIG. 23B, there was a complete loss of induction of both target genes in cells lacking HIF1β when exposed to BA or MBA. In contrast, our shRNA control exhibited an almost identical profile as the wild type treatments. FIG. 23C exhibits HIF1β depletion by approximately 84% in the T84 KD cells as compared with control. These findings confirm that through MBA exposure, HIF is stabilized and it is transcriptionally active. Furthermore, MBA allows a more prominent induction of HIF target genes compared to BA.

### Butyrate Derivatives Effects on Epithelial Cell Barrier Function

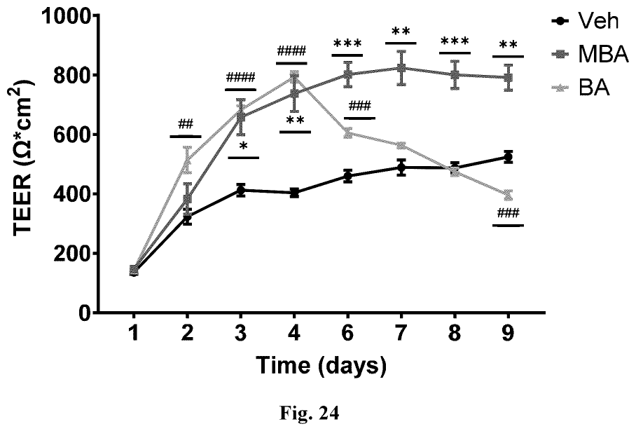

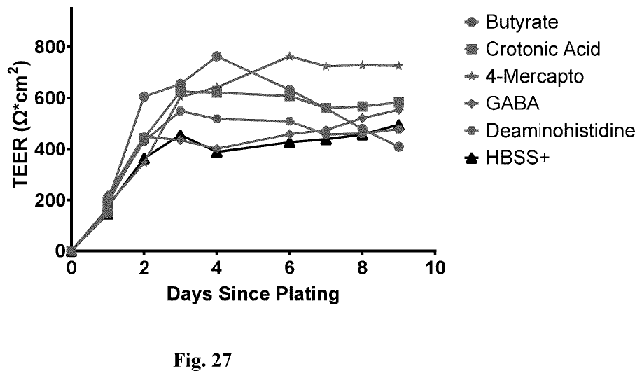

Studies have indicated that HIF orchestrates the regulation of various genes responsible for epithelial barrier function and barrier-adaptive responses. Therefore, we decided to study the effect of various butyrate derivatives in barrier function; in fact, sodium butyrate is an established pro-barrier factor. We studied epithelial barrier integrity through measurement of transepithelial electrical resistance (TEER), a typical assay for the quantitation of epithelial barrier strength. IEC c2bbe were exposed to butyrate derivatives (all at 5 mM) and TEERS were measured daily. Various derivatives demonstrated a considerable effect on barrier formation compared to vehicle (FIG. 27). Interestingly, both MBA and BA produce a significant increase in barrier (up to 800Ω*cm²), however, BA treated cells lose the integrity of their barrier over the course of the experiment in contrast to MBA exposed IECs (FIG. 24).

HIF Target Genes are Induced In-Vivo after Treatment with 4-Mercapto Butyrate

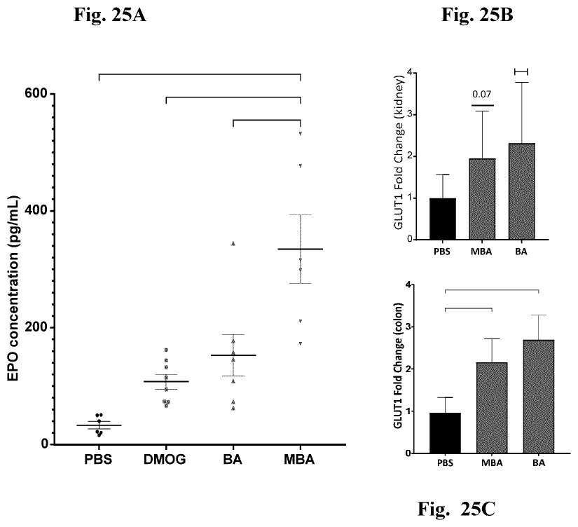

A typical physiologic response to hypoxia is the increase in red blood cell production. Naturally, HIF is in charge of this response by regulating cell-type specific gene expression that leads to increased levels of the hormone erythropoietin (EPO).[10] The study of EPO greatly contributed to the discovery of HIF which resulted in the 2019 Nobel Prize in Physiology or Medicine. We lastly explored the in-vivo effects of MBA in mediating induction of genes tightly regulated by HIF. C57BL/6 mice received one intraperitoneal injection of equimolar amounts of BA (73 mg/kg), MBA (80 mg/kg), an established in-vivo PHD inhibitor DMOG (117 mg/kg) [11, 12] and PBS (200 uL). After 24h of treatment, they were sacrificed and quickly exsanguinated to measure EPO levels in serum. MBA treated mice exhibited a significantly higher concentration of EPO compared to vehicle, DMOG and BA (FIG. 25A). Furthermore, we studied the induction of GLUT1, another classic HIF target responsible for the transport of various monosaccharides. Kidney and colon tissue were analyzed by real-time PCR to compare BA and MBA head-to-head. As observed in FIGS. 25B and C, both treatments resulted in an induction of GLUT1 in kidney and significantly increased in colon tissue. These findings support the ability of MBA to stabilize HIF in-vivo leading to the induction of classic target genes and transcription of a tightly regulated protein EPO.

1. A composition for use in stabilizing HIF comprising 4-Mercaptobutyrate.

2. The composition of claim 1, wherein said composition is used to treat intestinal disease in a patient in need.

3. A method of stabilizing HIF comprising administering to a subject in need a compound selected from the group consisting of Butyric acid, crotonic acid, 3-Chloro butyric acid, 2-Bromo butyric acid, 4-Mercapto butyric acid, GABA, 3-Phenyl butyric acid and combinations thereof.

4. A method of treating or preventing intestinal disease comprising administering an effective amount of a compound selected from the group consisting of Butyric acid, crotonic acid, 3-Chloro butyric acid, 2-Bromo butyric acid, 4-Mercapto butyric acid, GABA, 3-Phenyl butyric acid and combinations thereof.

5. (canceled)

6. The method of claim 4 wherein the subject has one or more symptoms of: Crohn's disease, ulcerative colitis, irritable bowel syndrome, inflammatory bowel disease, or gastrointestinal cancer.
